(19) United States
(12) Patent Application Publication
(10) Pub. No.: US 2013/0030293 A1
(43) Pub. Date: Jan. 31, 2013

(54) ULTRASOUND DIAGNOSTIC APPARATUS AND METHOD THEREOF

(71) Applicant: Shunichiro TANIGAWA, Tokyo (JP)

(72) Inventor: Shunichiro TANIGAWA, Tokyo (JP)

(21) Appl. No.: 13/560,250

(22) Filed: Jul. 27, 2012

(30) Foreign Application Priority Data
Jul. 28, 2011 (JP) 2011-165000

(51) Int. Cl. A61B 8/00

(52) U.S. Cl. 600/438

(57) ABSTRACT

An ultrasound diagnostic apparatus is provided. The ultrasound diagnostic apparatus includes a physical quantity calculating unit configured to calculate a physical quantity related to elasticity of biological tissue, based on echo signals obtained by transmission/reception of ultrasound to and from a subject, and a three-dimensional elastic image data generating unit configured to generate three-dimensional elastic image data by volume rendering processing that projects data related to the physical quantity in a three-dimensional region of the subject in a predetermined visual line direction to thereby obtain data of respective pixels on a projection plane, wherein the three-dimensional elastic image data generating unit is configured to obtain data corresponding to the number of data related to the physical quantity in a prescribed range of elasticity in the visual line direction as the data of the respective pixels.

### CROSS REFERENCE TO RELATED APPLICATIONS

This application claims the benefit of Japanese Patent Application No. 2011-165000 filed Jul. 28, 2011, which is hereby incorporated by reference in its entirety.

### BACKGROUND OF THE INVENTION

The present invention relates to an ultrasound diagnostic apparatus, and particularly to an ultrasound diagnostic apparatus for displaying elastic images each indicative of the hardness or softness of biological tissue, a method thereof, and a control program thereof

An ultrasound diagnostic apparatus, which combines a normal B-mode image and an elastic image indicative of the hardness or softness of biological tissue together and displays the result of combination, has been disclosed in, for example, Japanese Patent No. 3932482. In this type of ultrasound diagnostic apparatus, the elastic image is generated in the following manner. First, the transmission/reception of ultrasound is performed on biological tissue while deforming the biological tissue by repeating pressure by, for example, an ultrasound probe and its relaxation, thereby acquiring echoes. Then, a physical quantity related to the elasticity of the biological tissue is calculated based on data about the echoes, and the physical quantity is converted to color information to generate a colored elastic image. Incidentally, for example, distortion of the biological tissue or the like is calculated as the physical quantity related to the elasticity of the biological tissue.

Meanwhile, in Japanese Patent No. 3932482, the combined image obtained by combining the B-mode image and the elastic image together is a two-dimensional image. It is therefore difficult to grasp a stereoscopic form to be observed such as a tumor or the like. There has therefore been a demand for an ultrasound diagnostic apparatus which displays a three-dimensional elastic image capable of grasping a stereoscopic form to be observed.

Here, a mass in tissue is harder than normal tissue existing therearound. There is, however, also a case in which the entire inside of the mass is not hard uniformly and includes a partly soft portion. Displaying a three-dimensional elastic image on which the difference in elasticity in the interior of the mass has been reflected is thus effective in diagnosis. With the foregoing in view, there has been a demand for an ultrasound diagnostic apparatus capable of displaying a three-dimensional elastic image on which the difference in elasticity in the interior of an object to be observed in a predetermined range of elasticity has been reflected, a method thereof, and a control program thereof.

### BRIEF DESCRIPTION OF THE INVENTION

In one aspect, an ultrasound diagnostic apparatus is provided. The ultrasound diagnostic apparatus includes a physical quantity calculating unit which calculates a physical quantity related to elasticity of biological tissue, based on echo signals obtained by transmission/reception of ultrasound to and from a subject, and a three-dimensional elastic image data generating unit which generates three-dimensional elastic image data by volume rendering processing for projecting data related to the physical quantity in a three-dimensional region of the subject in a predetermined visual line direction to thereby obtain data of respective pixels on a projection plane, wherein the three-dimensional elastic image data generating unit obtains data corresponding to the number of data related to the physical quantity in a prescribed range of elasticity in the visual line direction as the data of the respective pixels.

In another aspect, an ultrasound diagnostic apparatus is provided. The ultrasound diagnostic apparatus includes a physical quantity calculating unit which calculates a physical quantity related to elasticity of biological tissue, based on echo signals obtained by transmission/reception of ultrasound to and from a subject; and a three-dimensional elastic image data generating unit which generates three-dimensional elastic image data by volume rendering processing for projecting data related to the physical quantity in a three-dimensional region of the subject in a predetermined visual line direction to thereby obtain data of respective pixels on a projection plane, wherein the three-dimensional elastic image data generating unit cumulatively calculates the data about the physical quantity in a prescribed range of elasticity in the predetermined visual line direction to obtain the data of the respective pixels.

According to one aspect described above, data corresponding to the number of data related to a physical quantity in a prescribed range of elasticity can be obtained as data of respective pixels on a two-dimensional projection plane at volume rendering processing. It is therefore possible to obtain a three-dimensional elastic image on which the difference in elasticity in the interior of a target to be observed has been reflected.

According to the invention of another aspect referred to above, data of respective pixels on a projection plane at volume rendering processing can be obtained by cumulatively calculating data related to a physical quantity in a prescribed range of elasticity in a predetermined visual line direction. It is therefore possible to obtain a three-dimensional elastic image on which the difference in elasticity in the interior of a target to be observed has been reflected.

Further objects and advantages will be apparent from the following description of exemplary embodiments as illustrated in the accompanying drawings.

### BRIEF DESCRIPTION OF DRAWINGS

FIG. 1 is a block diagram showing one example of a schematic configuration of an embodiment of an ultrasound diagnostic apparatus.

FIG. 2 is a block diagram illustrating a configuration of a display controller in the ultrasound diagnostic apparatus shown in FIG. 1.

FIG. 3 is an explanatory diagram depicting three sections orthogonal to one another.

FIG. 4 is a flowchart illustrating one example of an operation of the ultrasound diagnostic apparatus shown in FIG. 1.

FIG. 5 is a diagram showing one example of a display unit on which ultrasound images about three sections orthogonal to one another are displayed.

FIG. 6 is a diagram showing one example of the display unit in a state in which regions are set to the ultrasound images about the three sections orthogonal to each other.

FIG. 7 is a diagram for describing a three-dimensional region.

FIG. 8 is a diagram for describing a three-dimensional region.

FIG. 9 is a diagram for describing a three-dimensional region.

FIG. 10 is a diagram for describing the setting of a region.

FIG. 11 is a diagram showing one example of the display unit on which a three-dimensional elastic image is displayed together with the ultrasound images about the three sections orthogonal to one another.

FIG. 12 is a diagram for describing a prescribed range of elasticity.

FIG. 13 is an explanatory diagram of volume rendering processing.

FIG. 14 is a diagram showing a relationship between the number of color elastic image data and brightness.

FIG. 15 is an explanatory diagram of volume rendering processing.

FIG. 16 is a diagram showing a relationship between an added value of the inverse of gradation values and brightness in a second embodiment.

FIG. 17 is a diagram showing a relationship between an added value of gradation values and brightness in a first modification of the second embodiment.

FIG. 18 is a diagram showing another example of a relationship between an added value of gradation values and brightness in the first modification of the second embodiment.

FIG. 19 is a diagram illustrating a relationship between an added value of values obtained by squaring the inverse of gradation values, and brightness in a second modification of the second embodiment.

FIG. 20 is a diagram for describing the effect of the second modification of the second embodiment.

### DETAILED DESCRIPTION OF THE INVENTION

Exemplary embodiments will hereinafter be described in detail based on the accompanying drawings.

### First Embodiment

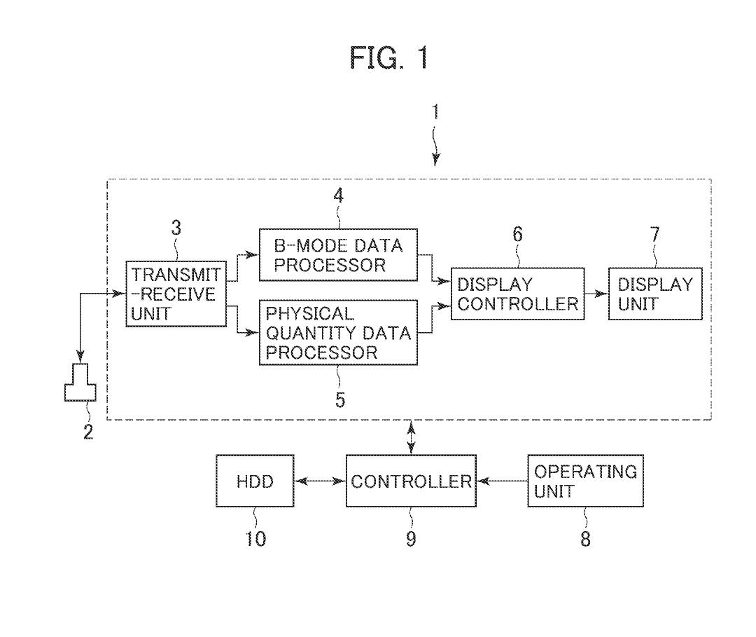

A first embodiment will first be explained based on FIGS. 1 through 15. An ultrasound diagnostic apparatus 1 shown in FIG. 1 is equipped with an ultrasound probe 2, a transmit-receive unit 3, a B-mode data processor 4, a physical quantity data processor 5, a display controller 6, a display unit 7, an operating unit 8, a controller 9 and an HDD (Hard Disk Drive) 10.

The ultrasound probe 2 transmits ultrasound to biological tissue and receives its echoes. The ultrasound probe 2 is an ultrasound probe which performs transmission/reception of ultrasound about a three-dimensional region to thereby make it possible to acquire volume data. More specifically, the ultrasound probe 2 includes a so-called mechanical 3D probe that mechanically performs scanning of a three-dimensional region, or a 3D probe that electronically performs scanning of a three-dimensional region. An elastic image is generated as will be described later, based on echo data acquired by performing the transmission/reception of the ultrasound while deforming the biological tissue by repeating pressure and relaxation in a state in which the ultrasound probe 2 is being brought into contact with surface of a subject or applying acoustic radiation pressure to the subject from the ultrasound probe 2.

The transmit-receive unit 3 drives the ultrasound probe 2 under a predetermined scan condition, based on a control signal outputted from the controller 9 to perform the scanning of the ultrasound every sound ray. The transmit-receive unit 3 performs signal processing such as phasing-adding processing on each echo signal received by the ultrasound probe 2. Echo data subjected to the signal processing by the transmit-receive unit 3 is outputted to the B-mode data processor 4 and the physical quantity data processor 5.

The B-mode data processor 4 performs B-mode processing such as logarithmic compression processing, envelope detection processing or the like on the echo data outputted from the transmit-receive unit 3 to thereby generate B-mode data. The B-mode data is outputted from the B-mode data processor 4 to the display controller 6.

The physical quantity data processor 5 generates data (physical quantity data) about a physical quantity related to the elasticity of each portion in the biological tissue, based on the echo data outputted from the transmit-receive unit 3 (physical quantity calculating function). As described in, for example, Japanese Patent Laid-Open No. 2008-126079, the physical quantity data processor 5 sets correlation windows to echo data different in time on the same sound ray position in one scanning plane. The physical quantity data processor 5 performs a correlation arithmetic operation between the correlation windows to calculate physical quantities related to the elasticity and thereby generates the physical quantity data. As the physical quantity related to the elasticity, may be mentioned distortion, for example.

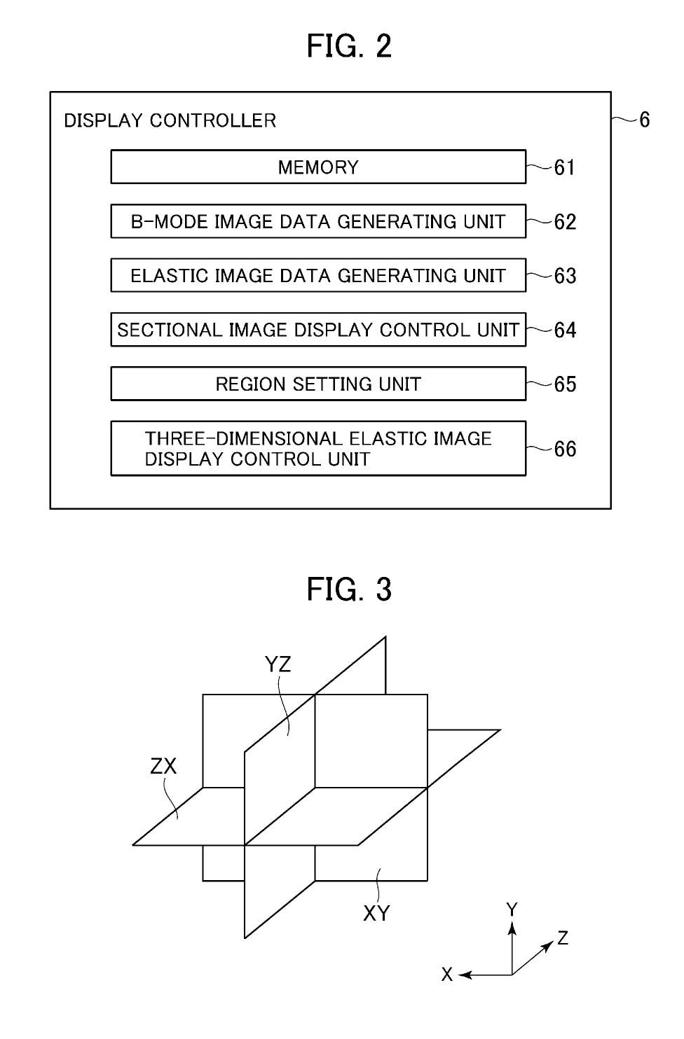

The display controller 6 is inputted with the B-mode data from the B-mode data processor 4 and the physical quantity data from the physical quantity data processor 5. As shown in FIG. 2, the display controller 6 has a memory 61, a B-mode image data generating unit 62, an elastic image data generating unit 63, a sectional image display control unit 64, a region setting unit 65 and a three-dimensional elastic image display control unit 66.

The memory 61 stores therein B-mode data and physical quantity data about respective scanning planes in a three-dimensional region subjected to the scanning of ultrasound by the ultrasound probe 2. Thus, the B-mode data and the physical quantity data stored in the memory 61 are volume data. The B-mode data and the physical quantity data are stored in the memory 61 as data set every sound ray.

The memory 61 is comprised of a semiconductor memory such as a RAM (Random Access Memory), a ROM (Read Only Memory), or the like. Incidentally, the B-mode data and the physical quantity data may be stored even in the HDD 10.

Assume now that data corresponding to echo data obtained by the transmission/reception of ultrasound and prior to being converted to B-mode image data and color elastic image data are raw data. The B-mode data and the physical quantity data stored in the memory 61 are raw data.

The B-mode image data generating unit 62 converts the B-mode data into B-mode image data BD having brightness information corresponding to the signal strength of echoes. The elastic image data generating unit 63 converts the physical quantity data into color elastic image data ED having color information corresponding to the distortion. Incidentally, the brightness information in the B-mode image data BD and the color information in the color elastic image data ED consist of predetermined gradations (e.g., 256 gradations). The data about the physical quantities in the exemplary embodiment contain data generated based on physical quantity data like the color elastic image data ED in addition to the physical quantity data itself.

The sectional image display control unit 64 causes the display unit 7 to display an ultrasound image G obtained by combining an elastic image EG and a B-mode image BG together. Described specifically, the sectional image display control unit 64 performs addition processing on the B-mode image data BD and the color elastic image data ED to combine them, thereby generating image data about a two-dimensional ultrasound image to be displayed on the display unit 7. This image data is displayed on the display unit 7 as a two-dimensional ultrasound image G obtained by combining a monochrome B-mode image BG and a color elastic image EG together. The elastic image EG is displayed in semitransparent form (in a see-through state of B mode image corresponding to the background).

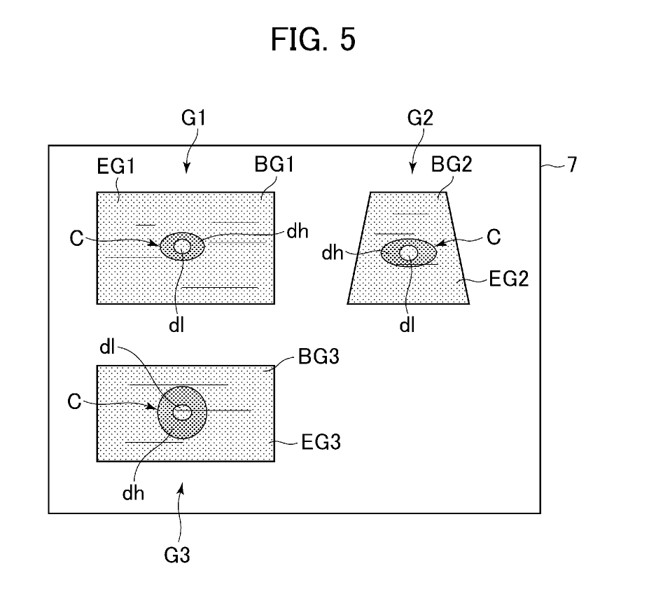

As shown in FIG. 3, the ultrasound image G corresponds to each of ultrasound images G1, G2 and G3 about three sections of a section XY, a section YZ and a section ZX orthogonal to each other (refer to FIG. 5 or the like). That is, the sectional image display control unit 64 combines the B-mode image data BD and the color elastic image data ED with the respect to the sections XY, YZ and ZX to generate image data and displays the ultrasound images G1 through G3.

The sectional image display control unit 64 may however display only an elastic image EG (corresponding to each of EG1 through EG3) based on the color elastic image data ED as the ultrasound image G (corresponding to each of G1 through G3).

The region setting unit 65 sets regions R1, R2 and R3 (refer to FIG. 6) to the ultrasound images G1 through G3 respectively. The region setting unit 65 sets the regions R1 through R3 based on an input given from the operating unit 8. The details thereof will be described later.

The three-dimensional elastic image display control unit 66 executes a three-dimensional elastic image data generating function for generating data (three-dimensional elastic image data) about a three-dimensional elastic image EG3D. The three-dimensional elastic image display control unit 66 causes the display unit 7 to display the three-dimensional elastic image EG3D, based on the three-dimensional elastic image data. The three-dimensional elastic image display control unit 66 generates the three-dimensional elastic image data with respect to a set three-dimensional region R3D specified based on the regions R1, R2 and R3 set to the ultrasound images G1 through G3 and displays the three-dimensional elastic image EG3D. The details thereof will be explained later.

The display unit 7 includes, for example, an LCD (Liquid Crystal Display), a CRT (Cathode Ray Tube) or the like. The operating unit 8 includes a keyboard and a pointing device or the like (not shown) for inputting instructions and information by an operator.

The controller 9 has a CPU (Central Processing Unit). The controller 9 reads a control program stored in the HDD 10 and executes functions at the respective parts of the ultrasound diagnostic apparatus 1 starting with the physical quantity calculating function, the three-dimensional elastic image data generating function, etc.

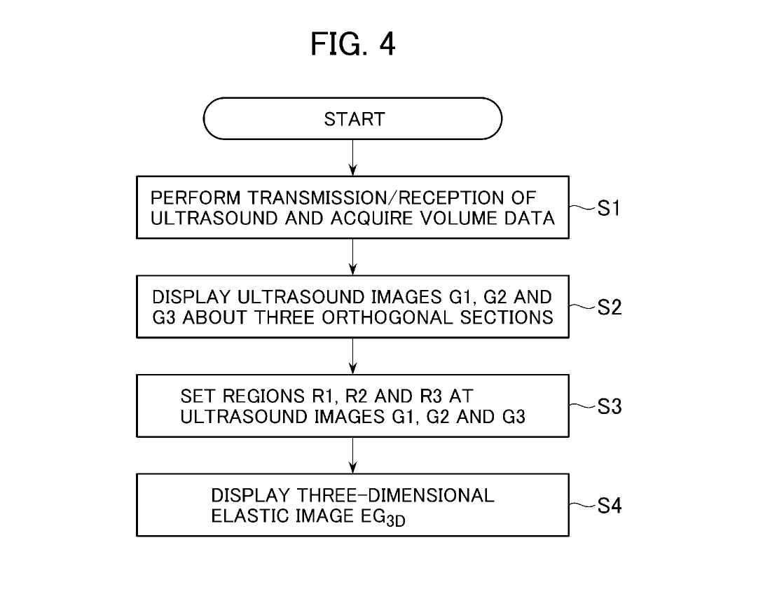

A description will now be made of the operation of the ultrasound diagnostic apparatus 1 according to the present embodiment, based on the flowchart of FIG. 4. At Step S1, the transmission/reception of ultrasound is first performed to acquire volume data. More specifically, the transmit-receive unit 3 transmits the ultrasound to biological tissue of a subject from the ultrasound probe 2 and thereby obtains its echo signals. At this time, the transmit-receive unit 3 performs the transmission/reception of ultrasound with respect to a three-dimensional region while deforming the biological tissue.

When the echo signals are obtained, the B-mode data processor 4 generates the B-mode data, and the physical quantity data processor 5 generates the physical quantity data. Further, the B-mode image data generating unit 62 generates B-mode image data BD, based on the B-mode data. The elastic image data generating unit 63 generates color elastic image data ED, based on the physical quantity data. Then, the B-mode image data BD and the color elastic image data ED about the three-dimensional region in which the scanning of ultrasound is done are stored in the memory 61 or the HDD 10.

Next, at Step S2, the sectional image display control unit 64 causes the display unit 7 to display ultrasound images G1 through G3 about sections XY, YZ and ZX (refer to FIG. 3) orthogonal to each other as shown in FIG. 5, based on the B-mode image data BD and the color elastic image data ED stored in the memory 61 or the HDD 10. The ultrasound image G1 is an image about the section XY and an image obtained by combining a B-mode image BG1 and an elastic image EG1. The ultrasound image G2 is an image about the section YZ and an image obtained by combining a B-mode image BG2 and an elastic image EG2. Further, the ultrasound image G3 is an image about the section ZX and an image obtained by combining a B-mode image BG3 and an elastic image BG3.

Each of the elastic images EG1 through EG3 is an image having a hue corresponding to the gradation value of the color elastic image data ED. In FIG. 5, the hues of the elastic images EG1 through EG3 are expressed in the density of dots. In each of the elastic images EG1 through EG3, a mass C to be observed consists of a portion dh higher in dot density than its periphery, and a portion d1 lower in dot density than the portion dh. The portion dh is a portion harder than peripheral normal tissue. The portion d1 is a portion softer than the portion dh.

Next, at Step S3, regions R1 through R3 are respectively set to the ultrasound images G1 through G3 (the elastic images EG1 through EG3) as shown in FIG. 6. Specifically, the operator performs an instruction input through the operating unit 8 in such a manner that the regions R1 through R3 are respectively set to desired positions in the ultrasound images G1 through G3. When the instruction input is given from the operating unit 8, the region setting unit 65 sets the regions R1 through R3.

The regions R1 through R3 are set to their corresponding masses C to be observed in the ultrasound images G1 through G3. With the setting of the regions R1 through R3, a three-dimensional region R_{3D} (not shown) to be targeted for generation of a three-dimensional elastic image EG_{3D} is specified.

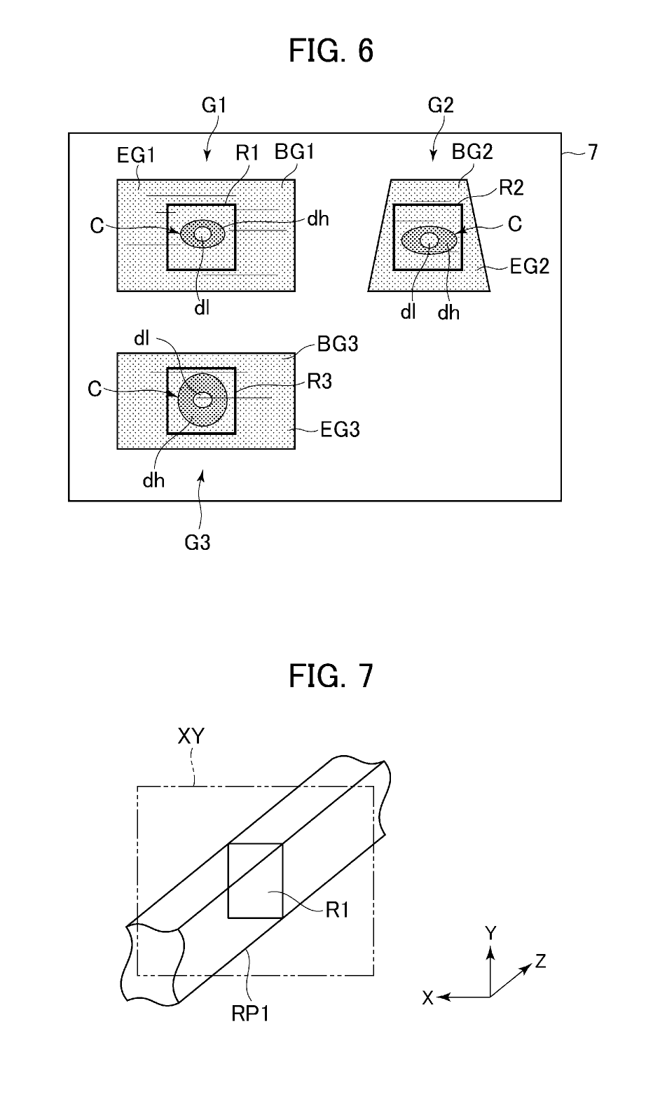

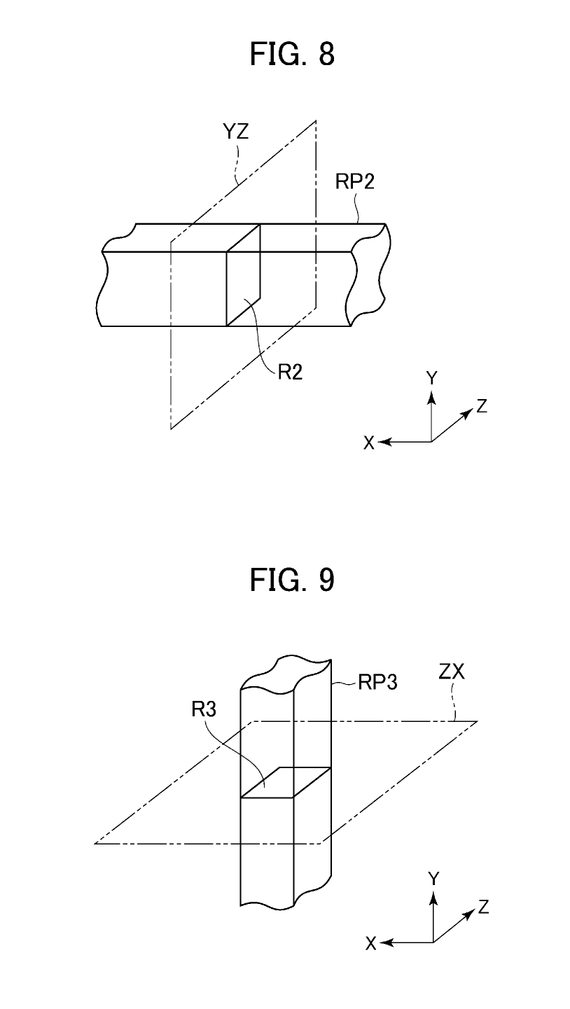

A description will now be made of specifying the three-dimensional region R_{3D} by the setting of the regions R1 through R3. When the region R1 about the section XY is set, a region RP1 of a square pillar in which the region R1 is assumed to be a section and a z-axis direction is assumed to be deep, is assumed as shown in FIG. 7. When the region R2 about the section YZ is set, a region RP2 of a square pillar in which the region R2 is assumed to be a section and an x-axis direction is assumed to be deep, is assumed as shown in FIG. 8. Further, when the region R3 about the section ZX is set, a region RP3 of a square pillar in which the region R3 is assumed to be a section and a y-axis direction is assumed to be deep, is assumed as shown in FIG. 9. A region in which the regions RP1, RP2 and RP3 are overlapped on one another, becomes the three-dimensional region R_{3D}.

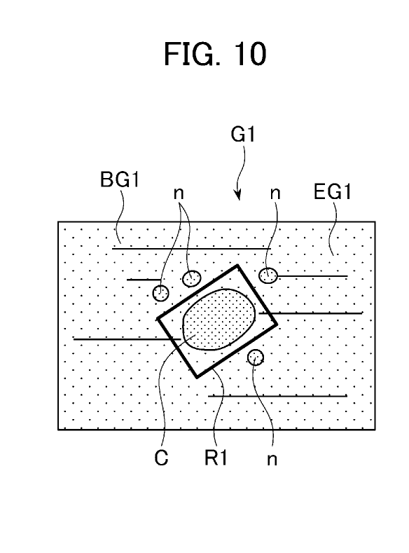

Incidentally, for example, when the ultrasound transmitted to the biological tissue does not reach the biological tissue sufficiently and when the condition of pressure and its relaxation to the biological tissue at the transmission/reception of the ultrasound is inappropriate, noise may occur in the corresponding elastic image EG. When such noise exists in the elastic image EG, the regions R1 through R3 may be set to avoid noise (noise is however not shown in FIG. 6). This will be explained in detail. An ultrasound image G1 is shown in FIG. 10. At an elastic image EG1 of the ultrasound image G1, signs n indicate noise portions displayed as the same elasticity as the mass C although being normal tissue. A region R1 is set to the periphery of the mass C to avoid the noise n. Setting the respective regions R1 through R3 in this manner makes it possible to display a three-dimensional elastic image EG3D at which it is easy to observe the mass C.

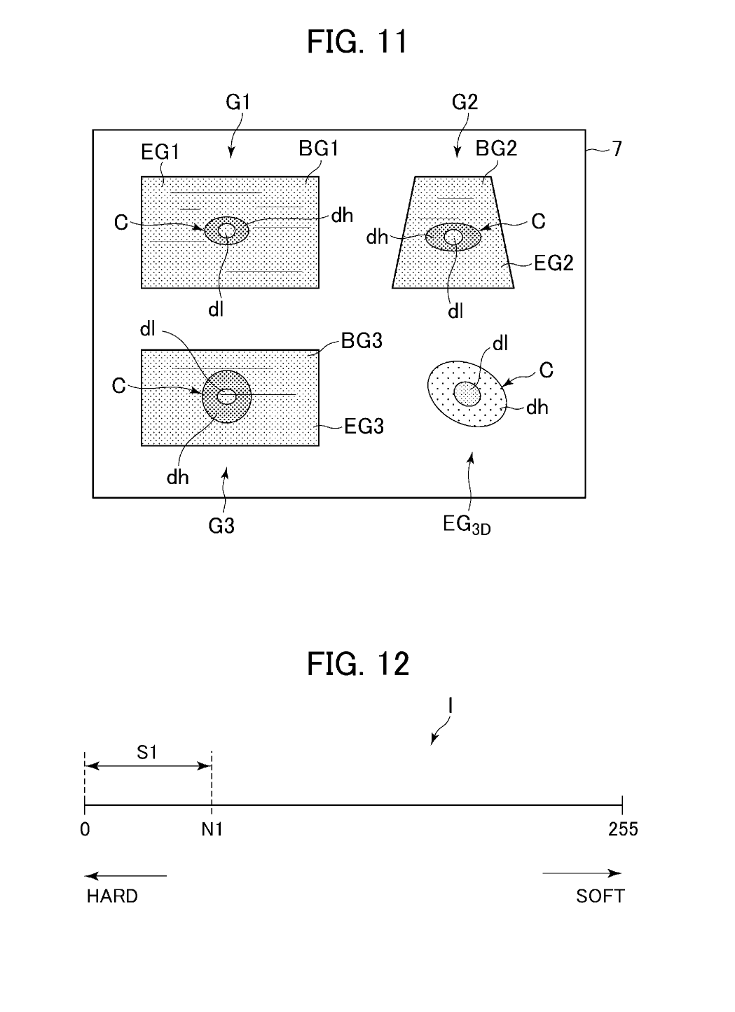

Next, at Step S4, the three-dimensional elastic image display control unit 66 generates three-dimensional elastic image data and displays a three-dimensional elastic image EG_{3D} as shown in FIG. 11. The three-dimensional elastic image EG_{3D} is displayed on the display unit 7 together with the ultrasound images G1 through G3. Incidentally, the regions R1 through R3 may or may not be displayed at the ultrasound images G1 through G3. The regions R1 through R3 are not displayed in FIG. 11.

A description will be made in detail of the generation of the three-dimensional elastic image data. The three-dimensional elastic image display control unit 66 generates three-dimensional elastic image data using preset color elastic image data ED in a prescribed range of elasticity set in advance, of color elastic image data (volume data) ED in the three-dimensional region R_{3D} specified based on the regions R1 through R3.

The prescribed range of elasticity will now be explained in detail. In the present example, the color elastic image data ED is data of 256 gradations ranging from 0 to 255. Thus, the physical quantity data is brought into gradation to 256 gradation display by the elastic image data generating unit 63 and results in color elastic image data ED.

The prescribed range of elasticity is set to gradation values of the 256 gradations. This will be explained in detail based on FIG. 12. A number line shown in FIG. 12 is assumed to be a number line indicative of 256 gradations ranging from gradation values 0 to 255. Assume that as the gradations values become small (on the gradation 0 side) in the number line 1, distortion is small and biological tissue is hard (the elasticity of the biological tissue is large), and as the gradation values become large (on the gradation 255 side), distortion is large and biological tissue is soft (the elasticity of the biological tissue is small).

The prescribed range of elasticity is set to a range S1 ranging from the gradation values 0 to N1 at the 256 gradations. Thus, the range S1 is set to the hard side, and the gradation value N1 becomes a gradation value at which the range S1 includes the elasticity of the portion dh in the mass C. On the other hand, the portion d1 is not contained in the range S1.

The prescribed range of elasticity may be set by the operator at the operating unit 8 or may be set as a default. The gradation value N1 may be inputted arbitrarily at the operating unit 8.

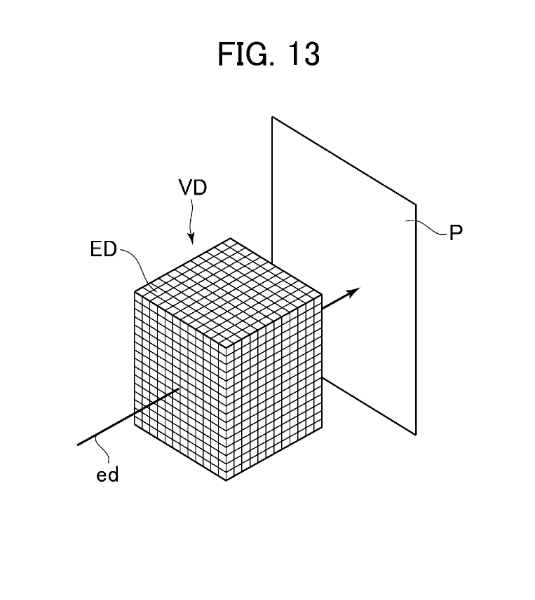

As shown in FIG. 13, the three-dimensional elastic image display control unit 66 performs volume rendering processing on volume data VD composed of color elastic image data ED in the three-dimensional region R_{3D} to generate three-dimensional elastic image data. The three-dimensional elastic image display control unit 66 performs volume rendering processing on volume data VD composed of the color elastic image data ED in the range S1, of the above volume data VD to generate three-dimensional elastic image data. Specifically, the three-dimensional elastic image display control unit 66 projects the color elastic image data ED of the range S1 in the three-dimensional region R_{3D} on a projection plane P in a predetermined visual line direction ed to thereby obtain data (pixel values) of respective pixels on the projection plane P. The pixel data on the projection plane P is of three-dimensional elastic image data.

The three-dimensional elastic image display control unit 66 acquires data about pixel values corresponding to the number of the color elastic image data ED of the range S1 in the visual line direction ed as the data of the respective pixels on the projection plane P.

Here, the three-dimensional elastic image EG_{3D} is an image which has a single hue and brightness different depending on the pixel values of the pixel data on the projection plane P. Alternatively, the three-dimensional elastic image EG_{3D} is an image which has an achromatic color (monochrome) and brightness different depending on the pixel values.

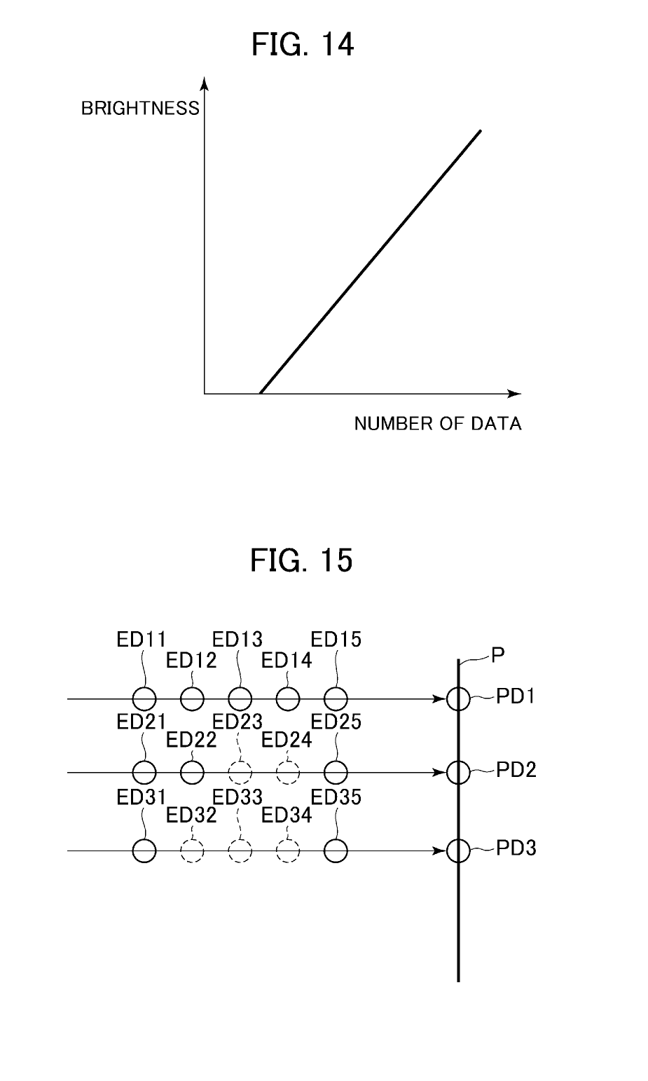

The data of the respective pixels on the projection plane P include information about the brightness of the three-dimensional elastic image EG3D. The brightness information depends on the number of the color elastic image data ED of the range S1. Specifically, the three-dimensional elastic image display control unit 66 obtains the data of the respective pixels on the projection plane P in such a manner that as shown in FIG. 14, as the number of the color elastic image data ED of the range S1 increases, the brightness of the three-dimensional elastic image EG3D becomes large, whereas as the number of the color elastic image data ED of the range S1 decreases, the brightness of the three-dimensional elastic image EG3D becomes small. This will be explained in detail based on FIG. 15. In FIG. 15, the three-dimensional elastic image display control unit 66 projects color elastic image data ED11, ED12, EDF13, ED14 and ED15 of the range S1 onto the projection plane P to obtain pixel data PD 1. The three-dimensional elastic image display control unit 66 projects color elastic image data ED21, ED22 and ED25 of the range S1 onto the projection plane P to obtain pixel data PD2. Further, the three-dimensional elastic image display control unit 66 projects color elastic image data ED31 and ED35 of the range S1 on the projection plane P to obtain pixel data PD3.

Incidentally, color elastic image data ED23, ED24, ED32, ED33 and ED34 indicated by broken lines in FIG. 15 are data other than the range S1.

The brightness indicated by the pixel values of the pixel data PD1 obtained based on the most data of the pixel data PD1, PD2 and PD3 is the highest. The brightness indicated by the pixel values of the pixel data PD3 obtained based on the least data thereof is the lowest.

Incidentally, assume that only some of the volume data in the three-dimensional region R3D are illustrated in FIG. 15. The number of the color elastic image data ED is for convenience of explanation. Pixel values of respective pixels may be obtained based on the number of data greater than the above number.

At the three-dimensional elastic image EG3D displayed on the display unit 7 based on the three-dimensional elastic image data generated in the above-described manner, the brightness becomes higher as the number of the color elastic image data ED of the range S1 in the visual line direction ed increases. Here, it means that as the number of the color elastic image data ED of the range S1 in the visual line direction ed increases, the number of portions large in the elasticity of the biological tissue in the visual line direction ed increases. Thus, the brightness of a part where portions hard in biological tissue are collected becomes large at the three-dimensional image EG3D. Specifically, the brightness of the portion dh is high and the brightness of the portion d1 is low. Thus, according to the ultrasound diagnostic apparatus of the present embodiment, the three-dimensional elastic image EG3D on which the internal difference in elasticity has been reflected, can be displayed with respect to a target to be observed such as the mass C.

Increasing the brightness of the part where the portions hard in biological tissue are collected at the three-dimensional elastic image EG_{3D} enables an easy grasp on where the hard portions are distributed. Thus, if reference is made to the three-dimensional elastic image EG_{3D}, it is possible to grasp a biopsy-needle sticking position easier when a biopsy needle is stuck into a harder portion at a mass, for example.

Incidentally, the three-dimensional elastic image EG_{3D} displayed on the display unit 7 may be set rotatably. It is thus possible to grasp much easier where the hard portion is distributed.

The graph shown in FIG. 14 is one example but is not limited to it. Although not shown in particular, for example, the number of the color elastic image data ED and the brightness may be placed in a nonlinear relationship.

### Second Embodiment

A second embodiment will next be explained. Incidentally, items different from those in the first embodiment will be explained in the following description.

In the present embodiment, the three-dimensional elastic image display control unit 66 performs a cumulative arithmetic operation or calculation on the color elastic image data ED of the range S1 in the visual line direction ed at the volume rendering processing to obtain data of respective pixels on the projection plane P. The data of the respective pixels are data having information about the brightness corresponding to cumulatively-calculated values. More specifically, the three-dimensional elastic image display control unit 66 adds the inverse of gradation values of color elastic image data ED in the visual line direction ed to obtain data of respective pixels.

This will be explained in detail. Assume that the gradation values of the color elastic image data ED 11, the color elastic image data ED 12, the color elastic image data ED 13, the color elastic image data ED 14, and the color elastic image data ED15 are “g11”, “g12”, “g13”, “g14” and “g15” respectively. Likewise, the gradation values of the color elastic image data ED21, ED22 and ED25 are respectively assumed to be “g21”, “g22” and “g25”. The gradation values of the color elastic image data ED31 and ED35 are respectively assumed to be “g31” and “g35”.

The three-dimensional elastic image display control unit 66 calculates an added value Add1 of the inverse of the gradation values of the color elastic image data ED11 through ED15, an added value Add2 of the inverse of the gradation values of the color elastic image data ED21, ED22 and ED25, and an added value Add3 of the inverse of the gradation values of the color elastic image data ED31 and ED35. That is, the three-dimensional elastic image display control unit 66 calculates the added values Add1 through Add3 in accordance with the following equations (1) through (3):

Add1=(1/g11)+(1/g12)+(1/g13)+(1/g14)+(1/g15) (1)

Add2=(1/g21)+(1/g22)+(1/g25) (2)

Add3=(1/g31)+(1/g35) (3)

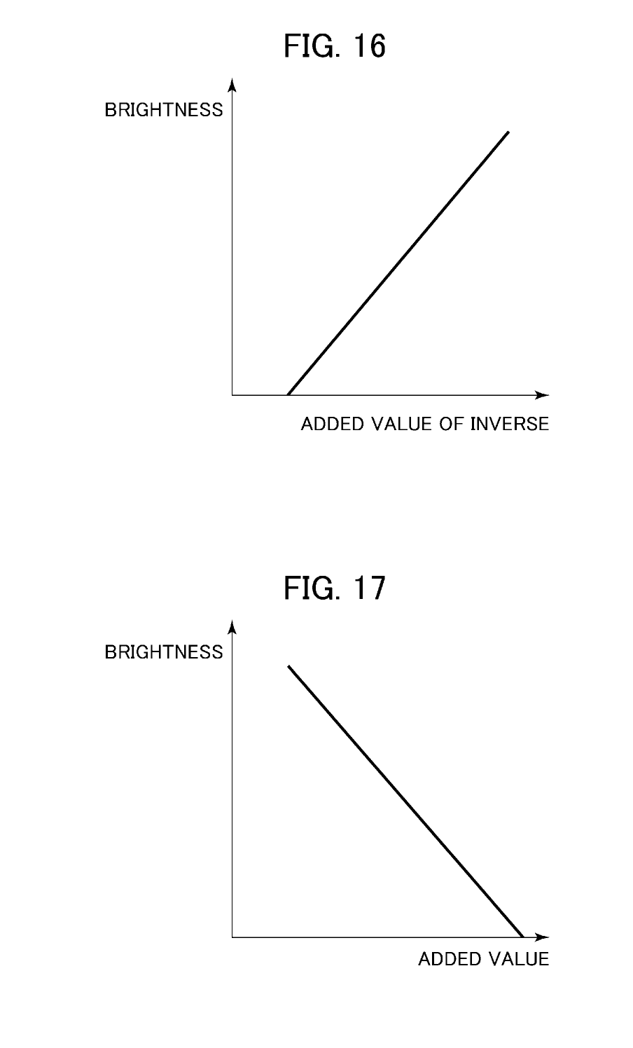

The three-dimensional elastic image display control unit 66 acquires the pixel data PD1, PD2 and PD3, based on the added values Add1 through Add3 in accordance with a graph shown in FIG. 16. That is, the three-dimensional elastic image display control unit 66 obtains the data of the respective pixels on the projection plane P in such a manner that as shown in FIG. 16, the brightness of the three-dimensional elastic image EG_{3D} becomes large as the added value of the inverse of the gradation values becomes large, whereas as the added value becomes small, the brightness of the three-dimensional elastic image EG_{3D} becomes small.

Now, the elasticity (elastic modulus of biological tissue) is large (the biological tissue is hard) as the gradation value becomes small. As the gradation value becomes large, the elasticity of the biological tissue is small (the biological tissue is soft). Thus, the smaller the gradation values of the respective color elastic image data ED in the visual line direction ed, the larger the added value (cumulatively-calculated value) of the inverse of the gradation values. The greater the number of the color elastic image data ED in the range S1 in the visual line direction ed, the larger the added value of the inverse of the gradation values. As the gradation values of the respective color elastic image data ED in the visual line direction ed become large, the added value of the inverse of the gradation values becomes small. As the number of the color elastic image data ED in the range S1 in the visual line direction ed becomes small, the added value of the inverse of the gradation values becomes small. The above shows that as the added value of the inverse of the gradation values becomes large, the elasticity of the biological tissue in the visual line direction in which the added value is obtained, is large, and that as the added value of the inverse of the gradation values becomes small, the elasticity of the biological tissue in the visual line direction in which the added value is obtained, is small. As described above, the larger the added value of the inverse of the gradation values, the greater the brightness of the three-dimensional elastic image EG_{3D}. The smaller the added value of the inverse of the gradation values, the lower the brightness of the three-dimensional elastic image EG_{3D}. Therefore, the data of the respective pixels can be obtained in such a manner that as the elasticity of the biological tissue becomes large, the brightness of the three-dimensional elastic image EG_{3D} becomes large. The data of the respective pixels can be obtained in such a manner that as the elasticity of the biological tissue becomes small, the brightness of the three-dimensional elastic image EG_{3D} becomes small.

According to the ultrasound diagnostic apparatus 1 of the present embodiment, the portion dh is greater than the portion d1 in the number of the color elastic image data ED in the range S1 as viewed in the visual line direction ed. For this reason, the portion dh becomes larger than the portion d1 in terms of the added value of the inverse of the gradation values of the color elastic image data ED in the range S1. Thus, in a manner similar to the first embodiment, the three-dimensional image EG_{3D} in which the portion dh is larger than the portion d1 in brightness can be displayed, and the three-dimensional elastic image EG_{3D} on which the internal difference in elasticity is reflected can be displayed with respect to the mass C.

In a manner similar to the first embodiment, the brightness of the part where the portions hard in biological tissue are collected is large at the three-dimensional elastic image EG_{3D}. It is therefore possible to easily grasp where the part hard in biological tissue is distributed.

Incidentally, the graph shown in FIG. 16 is merely one non-limiting example of the present embodiment.

Modifications of the second embodiment will next be explained. A first modification will first be described. The three-dimensional elastic image display control unit 66 may obtain the data of the respective pixels on the projection plane P in such a manner that as the elasticity of the biological tissue, which is indicated by the cumulatively calculated value (added value in the present example) of the color elastic image data ED in the range S1 as viewed in the visual line direction ed becomes large, the brightness of the three-dimensional elastic image EG_{3D} becomes large. For example, the three-dimensional elastic image display control unit 66 may add in the visual line direction, gradation values other than the inverse of gradation values of color elastic image data ED as they are. In this case, the three-dimensional elastic image display control unit 66 obtains data of respective pixels on the projection plane P, based on an added value of gradation values in accordance with a graph shown in FIG. 17. That is, the three-dimensional elastic image display control unit 66 obtains data of respective pixels on the projection plane P in such a manner that as shown in FIG. 17, the brightness of the three-dimensional elastic image EG_{3D} becomes large as the added value becomes small, and the brightness of the three-dimensional elastic image EG_{3D} becomes small as the added value becomes large.

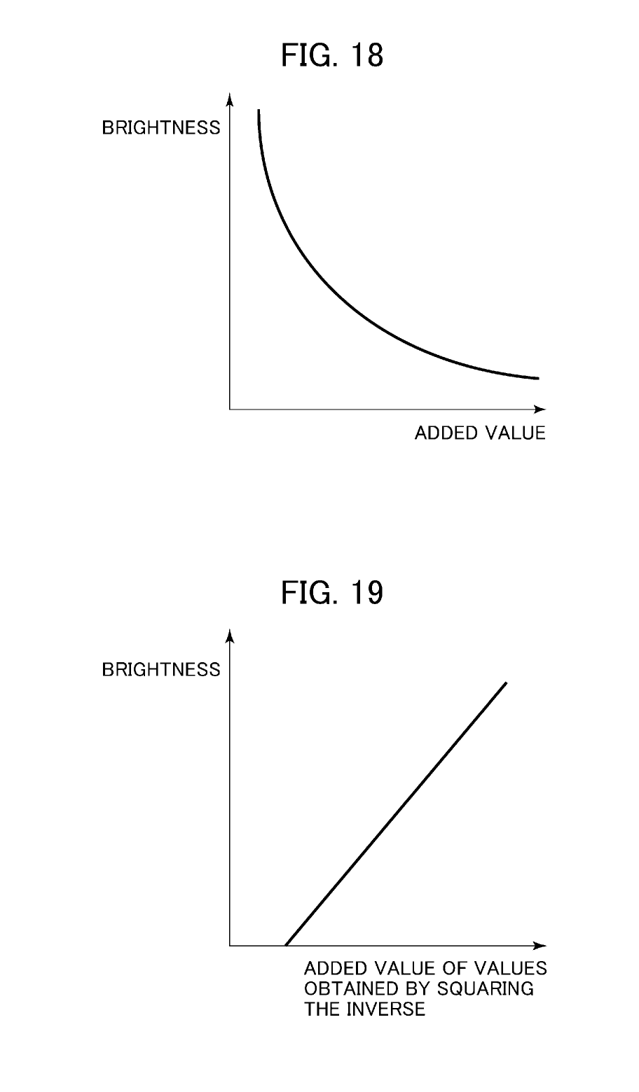

Incidentally, the graph shown in FIG. 17 is one example but is not limited to it. The three-dimensional elastic image display control unit 66 may obtain data of respective pixels on the projection plane P, based on an added value of gradation values in accordance with a graph shown in FIG. 18, for example.

A second modification will next be explained. The three-dimensional elastic image display control unit 66 may perform a cumulative arithmetic operation or calculation capable of obtaining a cumulatively calculated value at which color elastic image data indicating the elasticity of biological tissue is larger, i.e., color elastic image data smaller in gradation value has been emphasized. For example, the three-dimensional elastic image display control unit 66 may add values obtained by squaring the inverse of gradation values of the color elastic image data ED. More specifically, the three-dimensional elastic image display control unit 66 calculates an added value Add1′ of values obtained by squaring the inverse of gradation values of the color elastic image data ED11 through ED15, an added value Add2′ of values obtained by squaring the inverse of gradation values of the color elastic image data ED21, ED22 and ED25, and an added value Add3′ of values obtained by squaring the inverse of gradation values of the color elastic image data ED31 and ED35. That is, the three-dimensional image display control unit 66 calculates the added values Add1′ through Add3′ in accordance with the following equations (1) through (3):

Add1′=(1/g11)²+(1/g12)²+(1/g13)²+(1/g14)²+(1/g15)² (1)

Add2′=(1/g21)²+(1/g22)²+(1/g25)² (2)

Add3′=(1/g31)²+(1/g35)² (3)

In the second modification, the three-dimensional elastic image display control unit 66 obtains data of respective pixels on the projection plane P in accordance with a graph shown in FIG. 19, based on the resultant added values.

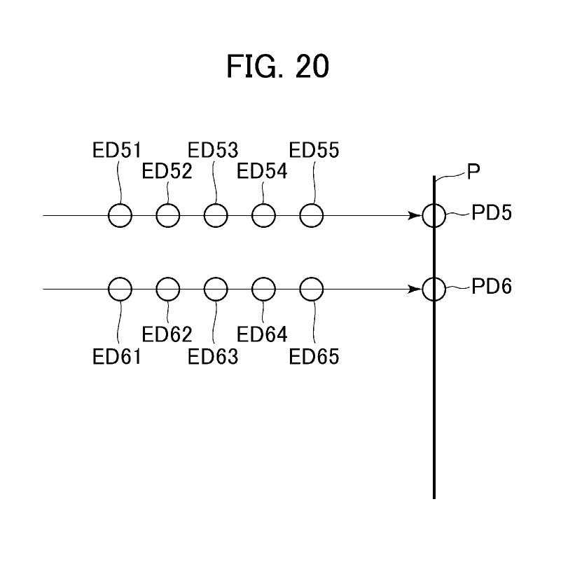

According to the second modification, there can be obtained an added value in which color elastic image data ED indicating the elasticity of the biological tissue is larger has been emphasized. This will be explained in detail based on FIG. 20. In FIG. 20, the three-dimensional elastic image display control unit 66 projects color elastic image data ED51, ED52, ED53, ED54 and ED55 onto the projection plane P to obtain pixel data PD5. The three-dimensional elastic image display control unit 66 projects color elastic image data ED61, ED62, ED63, ED64 and ED65 on the projection plane P to obtain pixel data PD6.

The gradation values of the color elastic image data ED51 through Ed55 are assumed to be “g51”, “g52”, “g53”, “g54” and “g55” respectively. The gradation values of the color elastic image data ED61 through ED65 are assumed to be “g61”, “g62”, “g63”, “g64” and “g65” respectively.

Assume that, for example, g51=3, g52=4, g53=1, g54=4 and g55=3, and g61=3, g62=3, g63=3, g64=3 and g65=3. Thus, the gradation value g53 of the color elastic image data ED53 is significantly smaller than other gradation values.

If the gradation values g51 through g55 and g61 through g65 are simply added, then the results of addition become g51+g52+g53+g54+g55=15, and g61+g62+g63+g64+g65=15. Therefore, the added values of both gradation values become equal to each other. Thus, when the gradation values are added in a simplistic form to obtain pixel data PD5 and PD6, the pixel data PD5 and PD6 become pixel values equal to each other.

However, an added value Add5′ of values obtained by squaring the inverse of the gradation values of the color elastic image data ED51, ED52, ED53, ED54 and ED55, and an added value Add6′ of values obtained by squaring the inverse of the gradation values of the color elastic image data ED61, ED62, ED63, ED64 and Ed65 are as follows:

Add5′=(⅓)²+(¼)²+1²+(¼)²+(⅓)²=97/72

Add6′=(⅓)²+(⅓)²+(⅓)²+(⅓)²+(⅓)²=5/9

Thus, the added value Add5′ becomes sufficiently larger than the added value Add6′ (Add5′>>Add6′). It is thus possible to obtain an added value at which the color elastic image data ED53 indicative of the elasticity of the biological tissue being larger has been emphasized.

Since the three-dimensional elastic image display control unit 66 obtains the pixel data in accordance with FIG. 19, the pixel data PD5 obtained based on the added value Add5′ is larger in brightness than the pixel data PD6 obtained based on the added value Add6′. As described above, the color elastic image data ED53 indicative of the elasticity of the biological tissue being larger can be reflected on the brightness of a three-dimensional elastic image.

Incidentally, the second modification of the second embodiments is not limited to the above arithmetic operation if a cumulative arithmetic operation or calculation is used which is capable of obtaining a cumulatively calculated value in which color elastic image data ED indicating the elasticity of biological tissue is larger has been emphasized.

Although exemplary embodiments have been described above, it is needless to say that the invention can be changed in various ways within the scope not departing from the gist thereof. In the above embodiments, for example, the prescribed range of elasticity is set with respect to the gradation values of the 256 gradations, but not limited to it. The prescribed range of elasticity may be set to physical quantities such as the value of the distortion, etc. In this case, volume rendering processing is performed with being aimed at the physical quantity data about the physical quantities in the prescribed range set to the prescribed range of elasticity so that three-dimensional elastic image EG_{3D} is generated and displayed. In this case, however, it is desired that the scanning of a three-dimensional region is electronically performed and echo data is acquired under a state in which the state of deformation of the biological tissue is preferably in the same state.

The physical quantity data generating unit 5 may calculate, as the physical quantity related to the elasticity of the biological tissue, a displacement based on the deformation of the biological tissue, its elastic modulus, etc. as an alternative to the distortion. A shear wave is generated in the biological tissue by applying acoustic radiation pressure to the biological tissue. The pascal (Pa) of the biological tissue may be calculated based on the velocity of the shear wave as a physical quantity about the elasticity of the biological tissue. Incidentally, the velocity of the shear wave can be calculated based on an echo signal of ultrasound. Further, the physical quantity about the elasticity of the biological tissue may be calculated by another known method.

Further, in the above embodiment, the three-dimensional elastic image data EG_{3D} is taken as the image having brightness corresponding to the pixel values on the projection plane P, but is not limited to it. The three-dimensional elastic image data EG_{3D} may be an image having a hue corresponding to each pixel value and opacity, etc.

Many widely different embodiments of the invention may be configured without departing from the spirit and the scope of the present invention. It should be understood that the present invention is not limited to the specific embodiments described in the specification, except as defined in the appended claims.

1. An ultrasound diagnostic apparatus comprising:
a physical quantity calculating unit configured to calculate a physical quantity related to elasticity of biological tissue, based on echo signals obtained by transmission/reception of ultrasound to and from a subject; and
a three-dimensional elastic image data generating unit configured to generate three-dimensional elastic image data by volume rendering processing that projects data related to the physical quantity in a three-dimensional region of the subject in a predetermined visual line direction to thereby obtain data of respective pixels on a projection plane,
wherein the three-dimensional elastic image data generating unit is configured to obtain data corresponding to the number of data related to the physical quantity in a prescribed range of elasticity in the visual line direction as the data of the respective pixels.

2. An ultrasound diagnostic apparatus according to claim 1, wherein the data of the respective pixels include information about the brightness of a three-dimensional image and include information about the brightness corresponding to the number of data related to the physical quantity in the prescribed range of elasticity in the visual line direction.

3. An ultrasound diagnostic apparatus according to claim 2, wherein the three-dimensional elastic image data generating unit is configured to obtain the data of the respective pixels in such a manner that as the number of the data corresponding to the physical quantity in the prescribed range of elasticity in the visual line direction increases, the brightness of the three-dimensional elastic image increases.

4. An ultrasound diagnostic apparatus comprising:
a physical quantity calculating unit configured to calculate a physical quantity related to elasticity of biological tissue, based on echo signals obtained by transmission/reception of ultrasound to and from a subject; and
a three-dimensional elastic image data generating unit configured to generate three-dimensional elastic image data by volume rendering processing that projects data related to the physical quantity in a three-dimensional region of the subject in a predetermined visual line direction to thereby obtain data of respective pixels on a projection plane,
wherein the three-dimensional elastic image data generating unit is configured to cumulatively calculate data related to the physical quantity in a prescribed range of elasticity in the predetermined visual line direction to obtain the data of the respective pixels.

5. An ultrasound diagnostic apparatus according to claim 4, wherein the data of the respective pixels include information about the brightness of a three-dimensional elastic image displayed based on the three-dimensional elastic image and include information about the brightness corresponding to a cumulatively calculated value of the data related to the physical quantity in the prescribed range of elasticity in the visual line direction.

6. An ultrasound diagnostic apparatus according to claim 5, wherein the three-dimensional elastic image data generating unit is configured to obtain data of respective pixels on the projection plane in such a manner that as the elasticity of the biological tissue indicated by the cumulatively calculated value becomes large, the brightness of a three-dimensional elastic image becomes large.

7. An ultrasound diagnostic apparatus according to claim 4, wherein the three-dimensional elastic image data generating unit is configured to perform the cumulative calculation in such a manner that a cumulated value in which data related to a physical quantity indicative of the elasticity of biological tissue being larger is emphasized is obtained.

8. An ultrasound diagnostic apparatus according to claim 4, wherein the cumulative calculation is an addition arithmetic operation.

9. An ultrasound diagnostic apparatus according to claim 1, wherein the data related to the physical quantity is gradated data obtained by gradating one of the data of the physical quantity and the physical quantity.

10. An ultrasound diagnostic apparatus according to claim 9, wherein the prescribed range of elasticity is set with respect to gradation values at the gradated data.

11. An ultrasound diagnostic apparatus according to claim 1, wherein the prescribed range of elasticity is set with respect to the physical quantity.

12. An ultrasound diagnostic apparatus according to claim 1, further comprising a sectional image display control unit configured to display elastic images about three sections orthogonal to one another which have been generated based on the physical quantity.

13. An ultrasound diagnostic apparatus according to claim 12, further comprising a region setting unit configured to set a predetermined region in each of the elastic images of the three sections,
wherein the three-dimensional elastic image data generating unit is configured to generate the three-dimensional elastic image data with respect to a three-dimensional region specified based on the region set by the region setting unit.

14. An ultrasound diagnostic apparatus according to claim 12, wherein the sectional image display control unit is configured to display the elastic images in the form of being combined with a B-mode image.

15. An ultrasound imaging method comprising:
calculating a physical quantity related to elasticity of biological tissue, based on echo signals obtained by transmission/reception of ultrasound to and from a subject;
generating three-dimensional elastic image data by volume rendering processing that projects data related to the physical quantity in a three-dimensional region of the subject in a predetermined visual line direction to thereby obtain data of respective pixels on a projection plane; and
obtaining data corresponding to the number of data related to the physical quantity in a prescribed range of elasticity in the visual line direction as the data of the respective pixels.

16. An ultrasound imaging method comprising:
calculating a physical quantity related to elasticity of biological tissue, based on echo signals obtained by transmission/reception of ultrasound to and from a subject;
generating three-dimensional elastic image data by volume rendering processing that projects data related to the physical quantity in a three-dimensional region of the subject in a predetermined visual line direction to thereby obtain data of respective pixels on a projection plane; and
cumulatively calculating data related to the physical quantity in a prescribed range of elasticity in the predetermined visual line direction to obtain the data of the respective pixels.

17. An ultrasound diagnostic apparatus according to claim 4, wherein the data related to the physical quantity is gradated data obtained by gradating one of the data of the physical quantity and the physical quantity.

18. An ultrasound diagnostic apparatus according to claim 17, wherein the prescribed range of elasticity is set with respect to gradation values at the gradated data.

19. An ultrasound diagnostic apparatus according to claim 4, wherein the prescribed range of elasticity is set with respect to the physical quantity.

20. An ultrasound diagnostic apparatus according to claim 4, further comprising a sectional image display control unit configured to display elastic images about three sections orthogonal to one another which have been generated based on the physical quantity.
